(19) United States
(12) Patent Application Publication
(10) Pub. No.: US 2007/0185042 A1
(43) Pub. Date: Aug. 9, 2007

(54) Sirna based methods for treating alzheimer's disease

(75) Inventors: Li-Huei Tsai, Cambridge, MA (US); Kenneth S. Kosik, Santa Barbara, CA (US)

Correspondence Address: FOLEY HOAG, LLP;PATENT GROUP (w/HUV HMV), 155 SEAPORT BLVD., BOSTON, MA, 02210-2600, US

(73) Assignee: The Brigham and Women's Hospital, Inc. President and Fellows of Harvard College

(21) Appl. No.: 10/567,537

(22) PCT Filed: Aug. 9, 2004
(86) PCT No.: PCT/US04/25633

§ 371 Date: Oct. 6, 2006

Related U.S. Application Data
(60) Provisional application No. 60/493,614, filed on Aug. 8, 2003.

(51) Int. Cl. A61K 48/00; C07H 21/02; C12N 5/08

(52) U.S. Cl. 514/44; 536/23.1; 435/455; 435/368

(57) ABSTRACT

The present invention relates to siRNA molecules derived from BACE1 and BACE2 genes. Accordingly, provided herein are siRNA molecules comprising a nucleotide sequence consisting essentially of a BACE1 or BACE2 gene. Also provided are methods for reducing the level of BACE1 protein in a cell. Further provided are methods for preparing a pharmaceutical composition comprising an siRNA with a pharmaceutically acceptable carrier.

### BACKGROUND

With the increase in life span globally, Alzheimer's disease (AD) featuring memory loss and dementia, poses a more serious problem for the society than ever. Despite an intensive search for therapeutic intervention, there is no drug that has proven effective in combating this devastating illness of the nervous system. The cause of AD is virtually unknown, although age is clearly the major risk factor. The pathology that is universal to all AD patients includes severe brain atrophy, neuronal loss, neurofibrillary tangles and senile plaques composed of aggregated β-amyloid (Aβ peptides [Whitehouse, 1985]. Interestingly, all known mutations that are associated with early onset AD enhance Aβ42 production [Price, 1998]. Thus, the amyloid cascade hypothesis has been put forward speculating that Aβ42 production plays an early and critical role in the pathogenesis of AD [Hardy, 1992] [Price, 1995] [Selkoe, 2000]. These amyloidogenic peptides, when aggregated, may elicit inflammatory and neurotoxic responses in the brain, which are believed to result in the clinical manifestations of AD.

Aβ peptides are generated from the sequential cleavage of the amyloid precursor protein (APP), a type I transmembrane protein, by an enzyme termed β-secretase in the ectodomain, and by an enzyme termed γ-secretase in the transmembrane region [FIG. 1A and Selkoe, 2003]. A C-terminal membrane bound fragment of 99 residues (C99) is produced by β-secretase cleavage of APP, which is subsequently cleaved by γ-secretase within the transmembrane domain to release Aβ peptides and APP intracellular domain (AICD). APP can also be cleaved by the α-secretase in the ectodomain which precludes its processing by the β-secretase pathway.

### SUMMARY

Provided herein are siRNA molecules comprising a nucleotide sequence consisting essentially of a sequence of a BACE1 gene, such as human BACE1, e.g., having a nucleotide sequence set forth in SEQ ID NO: 1. The nucleotide sequence of the BACE1 gene may consist of about 20 to 25 nucleotides. It may comprise SEQ ID NO: 3, 8, 13 or 16. It may also consist essentially of SEQ ID NO: 3, 8, 13 or 16.

Also provided are isolated nucleic acids encoding the sense strand, the antisense strand or both the sense and antisense strands of an siRNA molecule described herein and vectors and cells comprising such nucleic acids. Compositions comprising at least one or at least two siRNAs or nucleic acids encoding such are also encompassed.

Also provided are method for reducing the level of BACE1 protein in a cell, comprising administering into the cell, or contacting the cell with, an siRNA molecule or nucleic acid encoding such. The cell may comprise amyloid precursor protein (APP) and the method may reduce the level of β amyloid (Aβ) peptide in the cell relative to a cell to which an siRNA or nucleic acid was not administered. Methods described herein may also be used to protect a cell against stress, e.g., oxidative stress.

Also described are methods for preparing a pharmaceutical composition comprising combining an siRNA with a pharmaceutically acceptable carrier. The siRNAs may be used in methods for treating or preventing Alzheimer's disease in a subject, comprising administering to the subject a therapeutically effective amount of an siRNA, to thereby treat or prevent Alzheimer's disease. The administration of the siRNA may reduce the level of Aβ peptides. The method may comprise administering the siRNA into senile plaques.

### BRIEF DESCRIPTION OF THE FIGURES

FIG. 1A shows the generation of Aβ peptides from the amyloid precursor protein (APP).

FIG. 1B. Conserved sequences were chosen for designing siRNAs. cDNA sequences conserved between human, rat and mouse BACE1 but not BACE2 were chosen for generating siRNAs (SEQ ID NOS: 3-12 in the left column and SEQ ID NOS 13-20 in the right column, respectively in order of appearance).

FIG. 1C shows the nucleotide sequence of the open reading frame of human BACE1 (GenBank Accession number AF204943; SEQ ID NO: 1) and the location of siRNAs 1-4 (SEQ ID Nos: 3, 8, 13, and 16, respectively).

FIG. 2. Effects of siRNAs on human BACE1 and BACE2 expression in neuroblastoma CAD cells. (A) CAD cells were co-transfected with human APP, BACE1 analysis. Over 90% of BACE1 protein expression levels were knocked down by siRNA1 and siRNA2. (B) siRNA sequences chosen were specific to BACE1 and did not react with BACE2. CAD cells were co-transfected with human APP, BACE2 and siRNA for 24 hr. APP and BACE2 protein levels were analyzed.

FIG. 3. Suppression of endogeneous BACE1 expression in mouse primary cortical neurons. (A, B) Mouse primary cortical neurons were transfected with BACE1 siRNA or fluorescein RNA duplex as a control for 96 hr. Endogenous BACE1 levels were examined by Western blot analysis (the blots were reprobed with anti-Actin antibody to verify protein loading) (A) or immunocytochemistry staining (B). (C) BACE1 expression can be suppressed by siRNA sequence in a hairpin structure. CAD cells were co-transfected with human APP, BACE1 and pSilencer constructs containing hairpin BACE1 siRNA sequence for 24 hr. Negative control were provided by the manufacture. APP and BACE1 protein levels were examined by Western blot analysis. (D) Mouse primary cortical neurons were co-transfected with green fluorescence protein (GFP) and pSilencer construct containing siRNA sequence in a hairpin structure. BACE1 protein levels were examined by immunofluorescence staining.

FIG. 4. BACE1 suppression by siRNAs resulted in decreased C99/C89 generation and Aβ secretion. Wildtype mouse primary cortical neurons were transfected with siRNA for 72 hr and then infected with herpes simplex virus expressing human APP for additional 24 hr. Protein levels of APP full length and C-terminal fragments were detected by Western blot analysis using anti-APP antibodies (A). Conditioned media were collected and secreted Aβ measured by sandwitch ELISA (B, C).

FIG. 5. BACE1 suppression by siRNAs resulted in decreased C99/C89 generation and Aβ secretion in cortical neurons from APPsw Tg mice. APPsw Tg mouse primary cortical neurons were transfected with siRNA for 72 hr. Protein levels of BACE1, APP full length and C-terminal fragments were examined by Western blot analysis (A). Secreted Aβ from conditioned media were measured by sandwitch ELISA (B, C).

FIG. 6. Subcellular distribution of APP and Presenilin 1 was not changed after BACE1 suppression by siRNA1. Wildtype primary cortical neurons were transfected with siRNA1 for 72 hr. Cell homogenates were fractionated through an iodixanol step gradient and the distributions of BACE1, APP and Presenilin were analyzed.

FIG. 7. Pretreatment of BACE1 siRNA protected neurons from H₂O₂ induced cell death. Wild type primary cortical neurons were transfected with BACE1 siRNA1 for 72 hours (fluorescein RNA duplex as a control) and then treated with 10 μM H₂O₂ for 6 hours. Protein levels of BACE1 and APP full length and C-terminal fragments were detected by Western blot analysis. The blots were then reprobed with anti-Actin antibody to verify protein loading. (A). Cell viability was determined by MTT assay (B). Markedly reduced cell death was detected from neurons transfected with BACE1 siRNA (**p<0.001).

### DETAILED DESCRIPTION

BACE1 was identified as the β-secretase that cleaves APP in the ectodomain [Yan, 1999; Hussain, 1999; Sinha, 1999; Vassar, 1999]. It displays low level homology to the pepsin family of aspartyl proteases. A close homolog of BACE1, BACE2, exists in the mammalian genome that shares roughly 60% similarity with BACE1. BACE1 is expressed in all tissues with the highest expression in the brain. BACE1 protein is an intracellular type I transmembrane protein detected in the trans-Golgi network and endosomes. In brain samples of AD patients, the protein level and activity of BACE1 has been shown to be upregulated [Fukumoto, 2002; Holsinger, 2002; Yang, 2003]. The essential role of BACE1 in the generation of Aβ peptides is demonstrated by the finding that no Aβ peptides can be detected in mice with homozygous deletion of BACE1 [Roberds, 2001; Luo, 2001]. Interestingly, BACE1 null mice do not exhibit any developmental abnormality or outward behavioral phenotype.

The crystal structure of the ectodomain of BACE1 encompassing the catalytic domain complexed with a peptide inhibitor was reported by Hong and colleagues [Hong, 2000]. The overall structure of the enzyme is similar to that of the aspartyl proteases. One notable difference between BACE1 and most other aspartic proteases is that the active site is more open and less hydrophobic. This feature of BACE1 poses challenges for the development of small molecule inhibitors. As described herein, it has been discovered that BACE1 can be inhibited using small interfering RNA (siRNA) technology. As further described herein, BACE1 siRNA efficiently inhibits the generation of APP βCTFs and secretion of Aβ peptides in cultured neurons and in animal models. Furthermore, BACE siRNA also reduced Aβ production from neurons derived from transgenic mice harboring the Swedish APP mutant (APPsw). Thus, BACE1 siRNAs can be used to specifically inhibit the β-cleavage of APP and can be used, e.g., for treating Alzheimer's disease.

For convenience, the meaning of certain terms and phrases employed in the specification, examples, and appended claims are provided below. It is also to be understood that the terminology used herein is for the purpose of describing particular embodiments only and is not intended to be limiting.

Definitions

It must be noted that, as used in the specification and the appended claims, the singular forms “a,” “an” and “the” include plural referents unless the context clearly dictates otherwise.

When referring to a sequence that “consists of about” a certain number of nucleotides, this is intended to refer to a sequence that consists of the certain number of nucleotides plus or minus 20% or 10% of the number of nucleotides. For example, a sequence consisting of about 10 nucleotides refers to a sequence of 8, 9, 10, 11 or 12 nucleotides.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the nucleic acid, e.g., siRNA or nucleic acid encoding such, is released in a functional form.

“Essentially complementary” when referring to two nucleic acid strands refers to nucleic acid strands that are sufficiently complementary to allow hybridization of the two strands under the desired conditions. Accordingly, the two strands may be at least 90%, preferably at least 95% or 98% complementary. In other words, the two strands may differ in at most 5, 4, 3, 2 or 1 nucleotides, for example.

A “hairpin structure” when referring to the structure of a nucleic acid, refers to a single stranded nucleic acid in which two portions of the nucleic acid hybridize to each other to form the stem of a hairpin structure and a sequence located between the two portions forms a loop at one end of the stem.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes that contain certain mismatches, provided that the two strands are still forming a double stranded helix. “Stringent hybridization conditions” refers to hybridization conditions resulting in essentially specific hybridization.

“Inhibiting gene expression” refers to any action that results in decreased production of a polypeptide encoded by the gene or decreased levels of an RNA encoded by the gene. Inhibiting gene expression includes, e.g., inhibiting transcription, translation or degrading the DNA template or RNA encoded thereby.

“Non-human animals” include mammalians such as rodents, non-human primates, sheep, dog, cow, chickens, amphibians, reptiles, ovines, bovines, equines, canines, felines etc.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrase “nucleic acid corresponding to a gene” refers to a nucleic acid that can be used for detecting the gene, e.g., a nucleic acid which is capable of hybridizing specifically to the gene or complement thereof.

A nucleic acid is “operably linked” to another nucleic acid when it is placed into a functional relationship with another nucleic acid sequence. For example, a promoter or enhancer is operably linked to a coding sequence if it affects the transcription of the sequence. Generally, “operably linked” means that the DNA sequences being linked are contiguous. However, they can also be separated by other DNA sequences. Linking is accomplished by ligation at convenient restriction sites. If such sites do not exist, the synthetic oligonucleotide adaptors or linkers are used in accordance with conventional practice.

A nucleotide sequence is “perfectly complementary” or “perfectly matched” to another nucleotide sequence if each of the bases of the two sequences match, i.e., are capable of forming Watson-Crick base pairs. These terms also include the pairing of nucleoside analogs, such as deoxyinosine, nucleosides with 2-aminopurine bases, and the like, that may be employed. The term “complementary strand” is used herein interchangeably with the term “complement.” The complement of a nucleic acid strand can be the complement of a coding strand or the complement of a non-coding strand. “Essentially complimentary” refers to a duplex in which the two strands are sufficiently complimentary such as to be able to form a duplex under the desired conditions, e.g., in a cell. A mismatch in a duplex between a target polynucleotide and an oligonucleotide means that a pair of nucleotides in the duplex fails to undergo Watson-Crick bonding.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e., expression levels can be controlled).

“RNAi” stands for RNA-mediated interference.

“siRNA” stands for short (or small) interfering RNA. siRNAs comprise two sequences that are essentially complementary to each other so that they can hybridize under the desired conditions. The two sequences may be present on one strand or on two strands of nucleic acid. For example, the two sequences may be on one nucleic acid and separated by a spacer sequence that may form a loop when the two sequences interact.

The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

“Transcriptional regulatory sequence” is a generic term used throughout the specification to refer to DNA sequences, such as initiation signals, enhancers, promoters, and polymerase termination signals that induce or control transcription of protein coding sequences with which they are operably linked.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Exemplary Compositions

Provided herein are siRNA molecules comprising a nucleotide sequence consisting essentially of a sequence of a BACE1 gene, e.g., the sequence set forth in SEQ ID NO: 1. An siRNA molecule may comprise two strands, each strand comprising a nucleotide sequence that is at least essentially complementary to each other, one of which corresponds essentially to a sequence of a BACE1 gene. The sequence that corresponds essentially to a sequence of a BACE1 gene is referred to as the “sense BACE1 sequence” or more generally as a “sense target sequence” and the sequence that is essentially complementary thereto is referred to as the “antisense BACE1 sequence” or “antisense target sequence” of the siRNA. The sense and antisense target sequences may be from about 15 to about 30 consecutive nucleotides long; from about 19 to about 25 consecutive nucleotides; from about 19 to 23 consecutive nucleotides or about 19, 20, 21, 22 or 23 nucleotides long. The length of the sense and antisense sequences is determined so that an siRNA having sense and antisense target sequences of that length is capable of inhibiting expression of a target gene, preferably without significantly inducing a host interferon response.

The sense target sequence of an siRNA may be “essentially identical” or “substantially identical” to at least a part of the target gene. “Identity”, as known in the art, is the relationship between two or more polynucleotide (or polypeptide) sequences, as determined by comparing the sequences. In the art, identity also means the degree of sequence relatedness between polynucleotide sequences, as determined by the match between strings of such sequences. Identity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide sequences, the term is well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods commonly employed to determine identity between sequences include, but are not limited to those disclosed in Carillo, H., and Lipman, D., SLAM J. Applied Math., 48:1073 (1988). Preferred methods to determine identity are designed to give the largest match between the sequences tested. Methods to determine identity are codified in computer programs. Computer program methods to determine identity between two sequences include, but are not limited to, GCG program package (Devereux, J., et al., Nucleic Acids Research 12(1): 387 (1984)), BLASTP, BLASTN, and FASTA (Atschul, S. F. et al., 3. Molec. Biol. 215: 403 (1990)). Another software package well known in the art for carrying out this procedure is the CLUSTAL program. It compares the sequences of two polynucleotides and finds the optimal alignment by inserting spaces in either sequence as appropriate. The identity for an optimal alignment can also be calculated using a software package such as BLASTx. This program aligns the largest stretch of similar sequence and assigns a value to the fit. For any one pattern comparison several regions of similarity may be found, each having a different score. One skilled in the art will appreciate that two polynucleotides of different lengths may be compared over the entire length of the longer fragment. Alternatively small regions may be compared. Normally sequences of the same length are compared for a useful comparison to be made.

For example, the sense target sequence may be at least about 95%, 97%, 98%, 99% or 100% identical to a sequence of a BACE1 gene. Accordingly, in some embodiments, the sense target sequence differs from the sequence of a BACE1 gene in at most 5, 4, 3, 2, 1 or 0 nucleotides. The difference between the sense target sequence and the sequence of a BACE1 gene allowed is such that nucleic acids consisting of these two sequences are still capable of hybridizing under appropriate conditions (e.g., 400 mM NaCl, 40 mM PIPES pH 6.4, 1 mM EDTA, 50 or 70 degree C. hybridisation for 12-16 hours; followed by washing). Essentially the sequences may differ only to the extend that the difference does not significantly affect the ability of an siRNA comprising such a target sequence to inhibit the expression of a target gene, i.e., to mediate RNA interference. Accordingly, the degree of sequence differences permitted can be determined by determining the extend to which an siRNA having such a sense target sequence is capable of inhibiting the expression of a BACE1 gene.

The sense target sequence may be essentially or substantially identical to the coding or a non-coding portion, or combination thereof, of a target nucleic acid. For example, the sense target sequence may be essentially complementary to the 5′ or 3′ untranslated region, promoter, intron or exon of a target nucleic acid or complement thereof. It can also be essentially complementary to a region encompassing the border between two such gene regions.

The nucleotide base composition of the sense target sequence can be about 50% adenines (As) and thymidines (Ts) and 50% cytidines (Cs) and guanosines (Gs). Alternatively, the base composition can be at least 50% Cs/Gs, e.g., about 60%, 70% or 80% of Cs/Gs. Accordingly, the choice of sense target sequence may be based on nucleotide base composition. Regarding the accessibility of target nucleic acids by siRNAs, such can be determined, e.g., as described in Lee et al. (2002) Nature Biotech. 19:500. This approach involves the use of oligonucleotides that are complementary to the target nucleic acids as probes to determine substrate accessibility, e.g., in cell extracts. After forming a duplex with the oligonucleotide probe, the substrate becomes susceptible to RNase H. Therefore, the degree of RNase H sensitivity to a given probe as determined, e.g., by PCR, reflects the accessibility of the chosen site, and may be of predictive value for how well a corresponding siRNA would perform in inhibiting transcription from this target gene. One may also use algorithms identifying primers for polymerase chain reaction (PCR) assays or for identifying antisense oligonucleotides for identifying first target sequences.

The sense and antisense target sequences are preferably sufficiently complimentary, such that an siRNA comprising both sequences is able to inhibit expression of the target gene, i.e., to mediate RNA interference. For example, the sequences may be sufficiently complementary to permit hybridization under the desired conditions, e.g., in a cell. Accordingly, the sense and antisense target sequences may be at least about 95%, 97%, 98%, 99% or 100% identical and may, e.g., differ in at most 5, 4, 3, 2, 1 or 0 nucleotides.

Sense and antisense target sequences are also preferably sequences that are not likely to significantly interact with sequences other than the target nucleic acid or complement thereof. This can be confirmed by, e.g., comparing the chosen sequence to the other sequences in the genome of the target cell. Sequence comparisons can be performed according to methods known in the art, e.g., using the BLAST algorithm, further described herein. Of course, small scale experiments can also be performed to confirm that a particular first target sequence is capable of specifically inhibiting expression of a target nucleic acid and essentially not that of other genes.

siRNAs may also comprise sequences in addition to the sense and antisense sequences. For example, an siRNA may be an RNA duplex consisting of two strands of RNA, in which at least one strand has a 3′ overhang. The other strand can be blunt-ended or have an overhang. In the embodiment in which the RNA molecule is double stranded and both strands comprise an overhang, the length of the overhangs may be the same or different for each strand. In a particular embodiment, an siRNA comprises sense and antisense sequences, each of which are on one RNA strand, consisting of about 19-25 nucleotides which are paired and which have overhangs of from about 1 to about 3, particularly about 2, nucleotides on both 3′ ends of the RNA. In order to further enhance the stability of the RNA of the present invention, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly may also enhance the nuclease resistance of the overhang at least in tissue culture medium. RNA strands of siRNAs may have a 5′ phosphate and a 3′ hydroxyl group.

An siRNA may comprise a sense target sequence having a nucleotide sequence set forth in SEQ ID NO: 1. For example, an siRNA may be comprising, consisting or consisting essentially of nucleotides 175 or 176-194; 257 or 258-276; 826 or 827-845; 1237 or 1238-1256; or SEQ ID NO: 3, 8, 13, or 16. The sense target sequence may consist of SEQ ID NO: 3, 8, 13 or 16 and one or more additional nucleotides of SEQ ID NO: 1 at their 5′ or 3′ end. The sense target sequence may also consist of SEQ ID NO: 3, 8, 13 or 16 less one or more nucleotides at their 5′ or 3′ end. Sense target sequences may also consist of SEQ ID NO: 3, 8, 13 or 16 with one or more additional or fewer nucleotides at either end and comprising one or more nucleotide substitutions.

In one embodiment, an siRNA molecule comprises two strands of RNA forming a duplex. In another embodiment, an siRNA molecule consists of one RNA strand forming a hairpin loop, wherein the sense and antisense target sequences hybridize and the sequence between the two target sequences is a spacer sequence that essentially forms the loop of the hairpin structure. The spacer sequence may be any combination of nucleotides and any length provided that two complimentary oligonucleotides linked by a spacer having this sequence can form a hairpin structure, wherein at least part of the spacer forms the loop at the closed end of the hairpin. For example, the spacer sequence can be from about 3 to about 30 nucleotides; from about 3 to about 20 nucleotides; from about 5 to about 15 nucleotides; from about 5 to about 10 nucleotides; or from about 3 to about 9 nucleotides. The sequence can be any sequence, provided that it does not interfere with the formation of a hairpin structure. In particular, the spacer sequence is preferably not a sequence having any significant homology to the first or the second target sequence, since this might interfere with the formation of a hairpin structure. The spacer sequence is also preferably not similar to other sequences, e.g., genomic sequences of the cell into which the nucleic acid will be introduced, since this may result in undesirable effects in the cell.

A person of skill in the art will understand that when referring to a nucleic acid, e.g., an RNA, the RNA may comprise or consist of naturally occurring nucleotides or of nucleotide derivatives that provide, e.g., more stability to the nucleic acid. Any derivative is permitted provided that the nucleic acid is capable of functioning in the desired fashion. For example, an siRNA may comprise nucleotide derivatives provided that the siRNA is still capable of inhibiting expression of the target gene.

For example, siRNAs may include one or more modified base and/or a backbone modified for stability or for other reasons. For example, the phosphodiester linkages of natural RNA may be modified to include at least one of a nitrogen or sulphur heteroatom. Moreover, siRNA comprising unusual bases, such as inosine, or modified bases, such as tritylated bases, to name just two examples, can be used in the invention. It will be appreciated that a great variety of modifications have been made to RNA that serve many useful purposes known to those of skill in the art. The term siRNA as it is employed herein embraces such chemically, enzymatically or metabolically modified forms of siRNA, provided that it is derived from an endogenous template.

There is no limitation on the manner in which an siRNA may be synthesised. Thus, it may synthesized in vitro or in vivo, using manual and/or automated procedures. In vitro synthesis may be chemical or enzymatic, for example using cloned RNA polymerase (e.g., T3, T7, SP6) for transcription of a DNA (or cDNA) template, or a mixture of both. SiRNAs may also be prepared by synthesizing each of the two strands, e.g., chemically, and hybridizing the two strands to form a duplex. In vivo, the siRNA may be synthesised using recombinant techniques well known in the art (see e.g., Sambrook, et al., MOLECULAR CLONING; A LABORATORY MANUAL, SECOND EDITION (1989); DNA CLONING, VOLUMES I AND II (D. N Glover ed. 1985); OLIGONUCLEOTIDE SYNTHESIS (M. J. Gait ed, 1984); NUCLEIC ACID HYBRIDISATION (B. D. Hames & S. J. Higgins eds. 1984); TRANSCRIPTION AND TRANSLATION (B. D. Hames & S. J. Higgins eds. 1984); ANIMAL CELL CULTURE (R. I. Freshney ed. 1986); IMMOBILISED CELLS AND ENZYMES (IRL Press, 1986); B. Perbal, A PRACTICAL GUIDE TO MOLECULAR CLONING (1984); the series, METHODS IN ENZYMOLOGY (Academic Press, Inc.); GENE TRANSFER VECTORS FOR MAMMALIAN CELLS (J. H. Miller and M. P. Calos eds. 1987, Cold Spring Harbor Laboratory), Methods in Enzymology Vol. 154 and Vol. 155 (Wu and Grossman, and Wu, eds., respectively), Mayer and Walker, eds. (1987), IMMUNOCHEMICAL METHODS IN CELL AND MOLECULAR BIOLOGY (Academic Press, London), Scopes, (1987), PROTEIN PURIFICATION: PRINCIPLES AND PRACTICE, Second Edition (Springer-Verlag, N.Y.), and HANDBOOK OF EXPERIMENTAL IMMUNOLOGY, VOLUMES I-IV (D. M. Weir and C. C. Blackwell eds 1986). For example, bacterial cells can be transformed with an expression vector which comprises the DNA template from which the siRNA is to be derived.

If synthesized outside the mammalian cell, the siRNA may be purified prior to introduction into the cell. Purification may be by extraction with a solvent (such as phenol/chloroform) or resin, precipitation (for example in ethanol), electrophoresis, chromatography, or a combination thereof. However, purification may result in loss of siRNA and may therefore be minimal or not carried out at all. The siRNA may be dried for storage or dissolved in an aqueous solution, which may contain buffers or salts to promote annealing, and/or stabilisation of the RNA strands.

The double-stranded structure may be formed by a single self-complementary RNA strand or two separate complementary RNA strands. RNA duplex formation may be initiated either inside or outside the mammalian cell.

It is known that mammalian cells can respond to extracellular siRNA and therefore may have a transport mechanism for dsRNA (Asher et al, Nature 223 715-717 (1969)). Thus siRNA may be administered extracellularly into a cavity, interstitial space, into the circulation of a mammal, or introduced orally. Methods for oral introduction include direct mixing of the RNA with food of the mammal, as well as engineered approaches in which a species that is used as food is engineered to express the RNA, then fed to the mammal to be affected. For example, food bacteria, such as Lactococcus lactis, may be transformed to produce the dsRNA (see WO93/17117, WO97/14806). Vascular or extravascular circulation, the blood or lymph systems and the cerebrospinal fluid are sites where the RNA may be injected.

RNA may be introduced into the cell intracellularly. Physical methods of introducing nucleic acids may also be used in this respect. siRNA may be administered using the microinjection techniques described in Zernicka-Goetz,. et al. Development 124, 1133-1137 (1997) and Wianny, et al. Chromosoma 107, 430-439 (1998).

Other physical methods of introducing nucleic acids intracellularly include bombardment by particles covered by the siRNA, for example gene gun technology in which the siRNA is immobilised on gold particles and fired directly at the site of wounding. Thus, the invention provides the use of an siRNA in a gene gun for inhibiting the expression of a target gene. Further, there is provided a composition suitable for gene gun therapy comprising an siRNA and gold particles. An alternative physical method includes electroporation of cell membranes in the presence of the siRNA. This method permits RNAi on a large scale. Other methods known in the art for introducing nucleic acids to cells may be used, such as lipid-mediated carrier transport chemical-mediated transport, such as calcium phosphate, and the like. siRNA may be introduced along with components that perform one or more of the following activities: enhance RNA uptake by the cell, promote annealing of the duplex strands, stabilise the annealed strands, or otherwise increase inhibition of the target gene.

Any known gene therapy technique can be used to administer the RNA. A viral construct packaged into a viral particle would accomplish both efficient introduction of an expression construct into the cell and transcription of siRNA encoded by the expression construct. Thus, siRNA can also be produced inside a cell. Vectors, e.g., expression vectors that comprise a nucleic acid encoding one or the two strands of an siRNA molecule may be used for that purpose. Accordingly, provided herein are nucleic acids comprising a sense target sequence that is essentially identical or consists essentially of a sequence of a BACE1 gene, such as SEQ ID NO: 3, 8, 13 and 16. The nucleic acid may further comprise an antisense sequence that is essentially complementary to the sense target sequence. The nucleic acid may further comprise a spacer sequence between the sense and the antisense target sequence. The nucleic acid may further comprise a promoter for directing expression of the sense and antisense sequences in a cell, e.g., an RNA Polymerase II or III promoter and a transcriptional termination signal. The sequences may be operably linked.

In one embodiment a nucleic acid comprises an RNA coding region (e.g., sense or antisense target sequence) operably linked to an RNA polymerase III promoter. The RNA coding region can be immediately followed by a pol III terminator sequence, which directs termination of RNA synthesis by pol III. The pol III terminator sequences generally have 4 or more consecutive thymidine (“T”) residues. In a preferred embodiment, a cluster of 5 consecutive Ts is used as the terminator by which pol III transcription is stopped at the second or third T of the DNA template, and thus only 2 to 3 uridine (“U”) residues are added to the 3′ end of the coding sequence. A variety of pol III promoters can be used with the invention, including for example, the promoter fragments derived from H1 RNA genes or U6 snRNA genes of human or mouse origin or from any other species. In addition, pol III promoters can be modified/engineered to incorporate other desirable properties such as the ability to be induced by small chemical molecules, either ubiquitously or in a tissue-specific manner. For example, in one embodiment the promoter may be activated by tetracycline. In another embodiment the promoter may be activated by IPTG (lacI system).

siRNAs can be produced in cells by transforming cells with two nucleic acids, e.g., vectors, each nucleic acid comprising an expressing cassette, each expression cassette comprising a promoter, an RNA coding sequence (one being a sense target sequence and the other being an antisense target sequence) and a termination signal. Alternatively, a single nucleic acid may comprise these two expression cassettes. In yet another embodiment, a nucleic acid encodes a single stranded RNA comprising a sense target sequence linked to a spacer linked to an antisense target sequence. The nucleic acids may be present in a vector, such as an expression vector, e.g., a eukaryotic expression vector that allows expression of the sense and antisense target sequences in cells into which it is introduced.

Vectors for producing siRNAs are described, e.g., in Paul et al. (2002) Nature Biotechnology 29:505; Xia et al. (2002) Nature Biotechnology 20:1006; Zeng et al. (2002) Mol. Cell 9:1327; Thijn et al. (2002) Science 296:550; BMC Biotechnol. 2002 Aug. 28; 2(1):15; Lee et al. (2002) Nature Biotechnology 19: 500; McManus et al. (2002) RNA 8:842; Miyagishi et al. (2002) Nature Biotechnology 19:497; Sui et al. (2002) PNAS 99:5515; Yu et al. (2002) PNAS 99:6047; Shi et al. (2003) Trends Genet. 19(1):9; Gaudilliere et al. (2002) J Biol Chem. 277(48):46442; US2002/0182223; US 2003/0027783; WO 01/36646 and WO 03/006477. Vectors are also available commercially. For example, the pSilencer is available from Gene Therapy Systems, Inc. and pSUPER RNAi system is available from Oligoengine.

Also provided herein are compositions comprising one or more siRNA or nucleic acid encoding an RNA coding region of an siRNA. Compositions may be pharmaceutical compositions and comprise a pharmaceutically acceptable carrier. Compositions may also be provided in a device for administering the composition in a cell or in a subject. For example a composition may be present in a syringe or on a stent. A composition may also comprise agents facilitating the entry of the siRNA or nucleic acid into a cell.

Further provided herein are cells comprising an siRNA or nucleic acid encoding such. A cell can be a mammalian cell, e.g., a human cell.

A person of skill in the art will recognize that other methods for inhibiting gene expression that target specific regions of a gene or RNA can be used, e.g., micro RNAs (miRNAs). miRNAs are believed to function by inhibition of translation, as miRNAs are understood to function. It is likely that small RNAs generated as described herein which are perfectly complementary to the target DNA may inhibit gene expression by targeting the RNAs for degradation, whereas small RNAs which are not perfectly complementary to the target DNA may inhibit gene expression by inhibiting translation (Ambros et al. (2001) Cell 107:823 and Gaudilliere et al. (2002) J. Biol. Chem. (September 13)). Other small RNAs, e.g., double stranded RNAs, or single stranded RNAs, such as antisense RNAs, and hsRNAs, may also be used.

Exemplary Methods

Provided herein are methods for inhibiting the expression of a BACE1 gene, e.g., a human BACE1 gene, such as having SEQ ID NO: 1 or encoding a protein having SEQ ID NO: 2. The method may comprise contacting a cell or a cell extract with an amount of BACE1 siRNA sufficient to inhibit at least about 50%, 70%, 80%, 90%, 95%, 97%, 98%, or 99% expression of a BACE1 gene. Inhibiting the expression may include reducing the level of protein, e.g., by at least about 50%, 70%, 80%, 90%, 95%, 97%, 98%, or 99% relative to that in a cell or cell extract that was not contacted with a BACE1 siRNA. By reducing or inhibiting expression is also meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. By modulating expression of a target gene, e.g., a BACE1 gene, is meant altering, e.g., reducing, transcription/translation of a coding sequence, e.g., genomic DNA, mRNA etc., into a polypeptide, e.g., protein, product. The method may further comprise determining the level of BACE1 mRNA or protein, e.g., by methods well known in the art. Determining the level of inhibition of BACE1 gene expression or protein level can be determined as further described herein. Methods may also comprise contacting a cell with or introducing in a cell one or more nucleic acids encoding an siRNA.

Various methods for introducing nucleic acids into cells are known in the art. Exemplary methods include transfection, e.g., with calcium phosphate; electroporation; liposome based techniques, and use of viral vectors. Any type of plasmid and vector commonly used in the art can be used with the method of the invention. A plasmid that can be used may include elements that are necessary for replication of the plasmid in prokaryotic cells and elements that are necessary for selection of those prokaryotic cells including the plasmid with an insert relative to those that do not include a plasmid and those which contain an empty plasmid. For example, BlueScript (BS) may be used.

Any means for the introduction of the nucleic acids into cells, e.g., mammalian cells, may be used for the delivery of the various nucleic acids or constructs into the target cell. It may be desirable to introduce at least 5, 10, 25, 50, 100, or more copies of a nucleic acid. In one embodiment, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. A colloidal system may be a lipid-complexed or liposome-formulated DNA. Formulation of DNA, e.g., with various lipid or liposome materials, may then be effected using known methods and materials and delivered to the recipient cell or mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Nucleic acids of the invention can also be delivered via nanotecbnology.

In a preferred method of the invention, the nucleic acids are delivered using viral vectors. The nucleic acids may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of nucleic acids, e.g., exogenous genes, in vivo, particularly into humans. Such embodiments of the subject expression constructs are specifically contemplated for use in various in vivo and ex vivo gene therapy protocols.

In one embodiment, an siRNA or nucleic acid encoding an siRNA for inhibiting BACE1 expression is administered to a subject, such as a mammal, e.g., a human, non-human primate, mouse, rat, canine, feline, ovine, bovine, equine, or sheep. The subject may be a subject having a disease relating to an excessive activity of BACE1, such as due to excessive levels or activity of BACE1. The disease may also be a disease associated with excessive C99/C89 or Aβ peptide, e.g., Alzheimer's disease. The siRNA or nucleic acid encoding such is preferably administered locally in the area having excessive BACE1 level or activity or in an area comprising senile plaques associated with Alzheimer's disease. For example, in the latter disease, a therapeutically effective amount of a BACE1 siRNA or nucleic acid encoding such can be administered into the brain of a patient, e.g., at a location containing aggregated Aβ peptides, e.g., as further described herein. The method may comprise first identifying a subject having Alzheimer's disease. The method may also further comprise monitoring the effect of the therapy on the disease.

Also provided herein are methods for preparing a pharmaceutical composition comprising, e.g., combining a BACE1 siRNA or nucleic acid encoding such and pharmaceutically acceptable carrier.

Methods for protecting cells against stress are also encompassed herein. As described in the Examples, it has been shown that BACE1 siRNAs protect a cell against damage by H₂O₂. Accordingly, it is believed that inhibiting BACE1 expression leads to protection of cells from stress, such as oxidative stress; heatshock; or toxicity from or excess of certain compounds. Thus, siRNAs described herein may be used to protect cells from damage resulting from stress conditions. Exemplary methods include contacting cells with an effective amount of a BACE1 siRNA to make the cells more resistant to damage or cell death, e.g., apoptosis, from stress. For example, neurodegenerative disorders involving oxidative damage and subsequent neuronal cell loss may be treated as described herein. BACE1 siRNAs may also be administered during cell, tissue or organ transplantation, in particular, neuronal cell transplantation, to, e.g., alleviate the deleterious effects of cell loss, e.g., neuronal cell less, associated with oxidative injury or oxidative stress-induced apoptotic cell death.

BACE1 is expressed in different cell types. Diseases that are associated with abnormal BACE1 levels or activity in these cells may also be treated as described herein. For example, BACE1 is expressed in lens and inhibition of its expression is believed to protect from lens degenerative diseases (Li et al. Mol Vis. 2003 May 1; 9:179). Treatment regimes may be established by using, e.g., systemic oxidative stress animal models.

Alzheimer disease (AD) transgenic animals may be used for establishing treatment regimes or for testing variants of the siRNAs described herein.

Furthermore, BACE1 siRNAs can also be used in vitro, e.g., in cell culture medium to protect cells from stress related injury or damage. For example, siRNAs can be added to culture medium of cells, such as cells from cell lines or primary cultures of cells. BACE1 siRNAs may also be contacted with samples of cells or tissues, such as blood samples, to protect cells. Cells that are stored in large amounts, e.g., blood in blood banks, may also be protected from stress damage by including BACE1 siRNAs.

Pharmaceutical Compositions

In another aspect, an siRNA or nucleic acid encoding such can be used in the production of an agent, e.g. a medicament, for inhibiting the expression of a BACE1 gene in a mammalian cell. The medicament may be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms. The pharmaceutical composition may be adapted for administration by any appropriate route, for example by topical, parenteral (including subcutaneous, intramuscular, intravenous or intradermal), oral (including buccal or sublingual), rectal, or nasal route. Such compositions may be prepared by any method known in the art of pharmacy, for example by admixing the active ingredient with the carrier(s) or excipient(s) under sterile conditions.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilised) condition requiring only the addition of the sterile liquid carried, for example water for injections, immediately prior to use. Extemporaneous injection solutions and suspensions may be prepared from sterile powders, granules and tablets.

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc.

For the preparation of solutions and syrups, excipients which may be used include for example water, polyols and sugars. For the preparation of suspensions oils (e.g. vegetable oils) may be used to provide oil-in-water or water in oil suspensions.

Pharmaceutical compositions adapted for nasal administration wherein the carrier is a solid include a coarse powder having a particle size for example in the range 20 to 500 μm which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient. Pharmaceutical compositions adapted for administration by inhalation include fine particle dusts or mists which may be generated by means of various types of metered dose pressurised aerosols, nebulizers or insufflators.

In one embodiment, an siRNA or nucleic acid encoding such is administered by direct injection into cerebrospinal fluid, or by stereotactic intracerebral inoculation into the hippocampus. In addition, some viral vectors, such as adenovirus, can be transported in a retrograde manner from the point of injection (Ridoux, V., et al., Brain Res. 648:171-175 (1994); Kuo, H., et al., Brain Res. 24:31-38 (1995)). Other routes of administration include nasal inhalation (Draghia, R., Gene Ther. 2:418-423 (1995)) and injection into the carotid artery after disruption of the blood brain barrier (Doran, S. E., et al., Neurosurgery 36:965-970 (1995); Muldoon, L. L., Am. J. Pathol. 147:1840-1851 (1995)).

When necessary, the pharmaceutical composition may be prepared so that the therapeutic agent, e.g., an siRNA or nucleic acid encoding such, passes through the blood-brain barrier. One way to accomplish transport across the blood-brain barrier is to couple or conjugate the therapeutic agent to a secondary molecule (a “carrier”), which is either a peptide or a non-proteinaceous moiety. The carrier is selected such that it is able to penetrate the blood-brain barrier. Examples of suitable carriers are pyridinium, fatty acids, inositol, cholesterol, and glucose derivatives. Alternatively, the carrier can be a compound which enters the brain through a specific transport system in brain endothelial cells, such as transport systems for transferring insulin, or insulin-like growth factors I and II. This combination of therapeutic agent and carrier may be called a prodrug. Upon entering the central nervous system, the prodrug may remain intact or the chemical linkage between the carrier and therapeutic agent may be hydrolyzed, thereby separating the carrier from the therapeutic agent See generally U.S. Pat. No. 5,017,566 to Bodor.

An alternative method for transporting the therapeutic agent across the blood-brain barrier is to encapsulate the carrier in a lipid vesicle such as a microcrystal or liposome. Such lipid vesicles may be single or multi-layered, and encapsulate the therapeutic agent either in the center thereof or between the layers thereof. Such preparations are well known. For example, PCT Application WO 91/04014 of Collins et al. describes a liposome delivery system in which the therapeutic agent is encapsulated within the liposome, and the outside layer of the liposome has added to it molecules that normally are transported across the blood-brain barrier. Such liposomes can target endogenous brain transport systems that transport specific ligands across the blood-brain barrier, including but not limited to, transferring insulin, and insulin-like growth factors I and II. Alternatively, antibodies to brain endothelial cell receptors for such ligands can be added to the outer liposome layer. U.S. Pat. No. 4,704,355 to Bernstein describes methods for coupling antibodies to liposomes.

Another method of formulating the therapeutic agent to pass through the blood-brain barrier is to prepare a pharmaceutical composition as described above, wherein the therapeutic agent is encapsulated in cyclodextrin. Any suitable cyclodextrin which passes through the blood-brain barrier may be employed, including .beta.-cyclodextrin, .gamma.-cyclodextrin, and derivatives thereof. See generally U.S. Pat. No. 5,017,566 to Bodor; U.S. Pat. No. 5,002,935 to Bodor; U.S. Pat. No. 4,983,586 to Bodor.

Another method of passing the therapeutic agent through the blood-brain barrier is to prepare and administer a pharmaceutical composition as described above, with the composition further including a glycerol derivative as described in U.S. Pat. No. 5,153,179 to Eibl.

An alternative method of delivering the therapeutic agent to the brain is to implant a polymeric device containing the agent, which device is able to provide controlled release delivery of the agent to the brain for an extended period after implantation. Examples of such implantable polymeric devices are described in U.S. Pat. No. 5,601,835 to Sabel, and in U.S. Pat. No. 5,846,565, to Brem.

Pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain other therapeutically active agents in addition to the therapeutics described herein.

Dosages of the substance of the present invention can vary between wide limits, depending upon the disease or disorder to be treated, the age and condition of the individual to be treated, etc. and a physician will ultimately determine appropriate dosages to be used. This dosage may be repeated as often as appropriate. If side effects develop the amount and/or frequency of the dosage can be reduced, in accordance with normal clinical practice.

The present invention may be used alone or as a component of a kit having at least one of the reagents necessary to carry out the in vitro or in vivo introduction of siRNA or nucleic acids encoding such to subjects. Exemplary components are the siRNA and a vehicle that promotes introduction of the siRNA. Such a kit may also include instructions to allow a user of the kit to practice the invention.

The present invention is further illustrated by the following examples, which should not be construed as limiting in any way. The contents of all cited references including literature references, issued patents, published and non published patent applications and GenBank Accession numbers as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Example 1

### siRNAs decrease BACE1 Expression

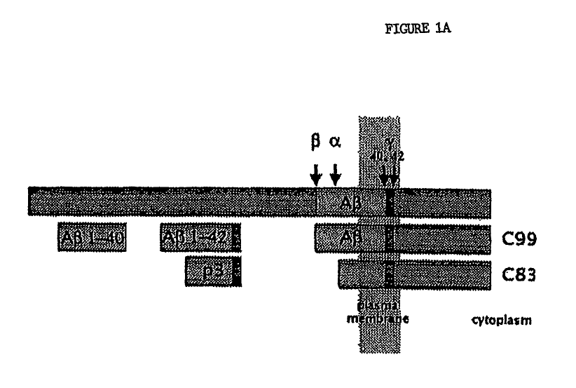

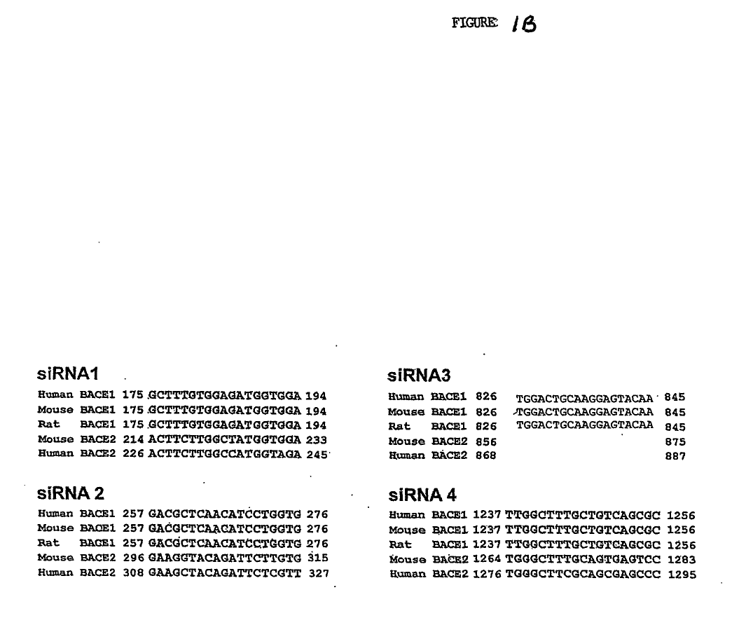

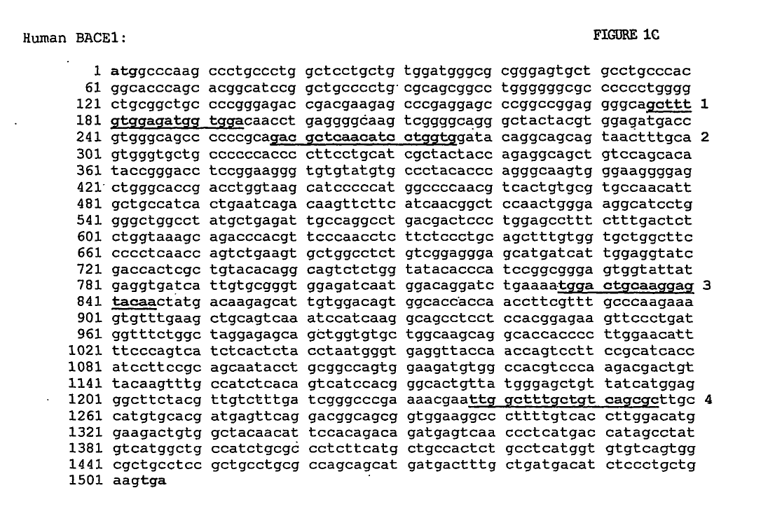

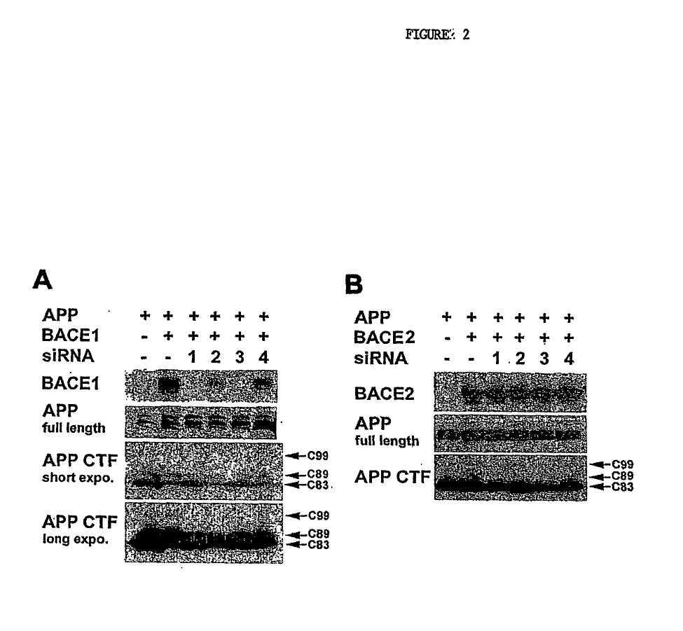

We chose 19 nt mRNA sequences that are conserved between human, rat and mouse BACE1, but not BACE2 as target sequences and designed four specific siRNAs against these target sequences (FIG. 1). To test the specificity of these siRNAs, we co-transfected a neuroblastoma cell line, CAD cells with BACE1 siRNA, human BACE1 or BACE2 cDNA and human APP cDNA. A fluorescein RNA duplex (non-silencing) that does not overlap with any known mammalian genome sequence was used as negative control. As shown in FIG. 2A, siRNA1 proved most efficient in knocking down BACE1 expression with an efficiency of 99% after 24 hours of treatment. siRNA3 also reduced BACE1 protein level by about 90%, whereas siRNA4 only marginally affected BACE1 expression with about 50% reduction.

### Example 2

### BACE1 siRNAs Reduce the Level of C89/C99

When measuring the level of Aβ peptides in Example 1, it was discovered that the levels of C89/C99, two major BACE1 cleavage products of APP, were also decreased. In contrast, none of the four sequences affected BACE2 expression (FIG. 2B). These observations demonstrate that recombinant BACE1 expression can be knocked down using specific siRNAs.

### Example 3

### siRNAs Decrease Endogenous BACE1 in Primary Neurons

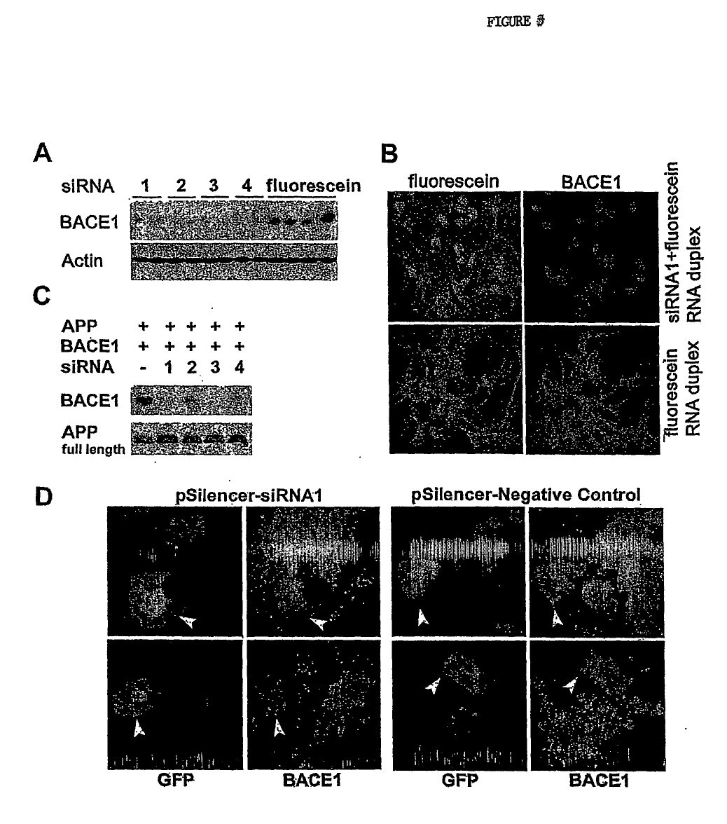

We next sought to investigate whether BACE1 siRNAs also affect the expression of endogenous BACE1 in mouse primary cortical neurons. Dissociated mouse cortical neurons were treated with the 4 siRNAs and BACE1 levels evaluated 72 hours after treatment. We observed a 70% reduction of BACE1 in siRNA treated neurons, but not in control fluorescein RNA treated neurons (FIG. 3A). The effect of siRNA to down-regulate BACE1 could also be demonstrated by immunocytochemistry. We co-transfected mouse primary cortical neurons with BACE1 siRNA and fluorescein RNA duplex as transfection indicator. We noticed that over 90% of neurons were fluorescein positive, indicating that siRNA were efficiently introduced into primary cortical neurons (FIG. 3B). In addition, the expression of BACE1 was reduced up to 50% by the cognate BACE1 siRNA in most the neurons.

### Example 4

### BACE1 Expression is Inhibited with Vectors Encoding BACE1 siRNAs

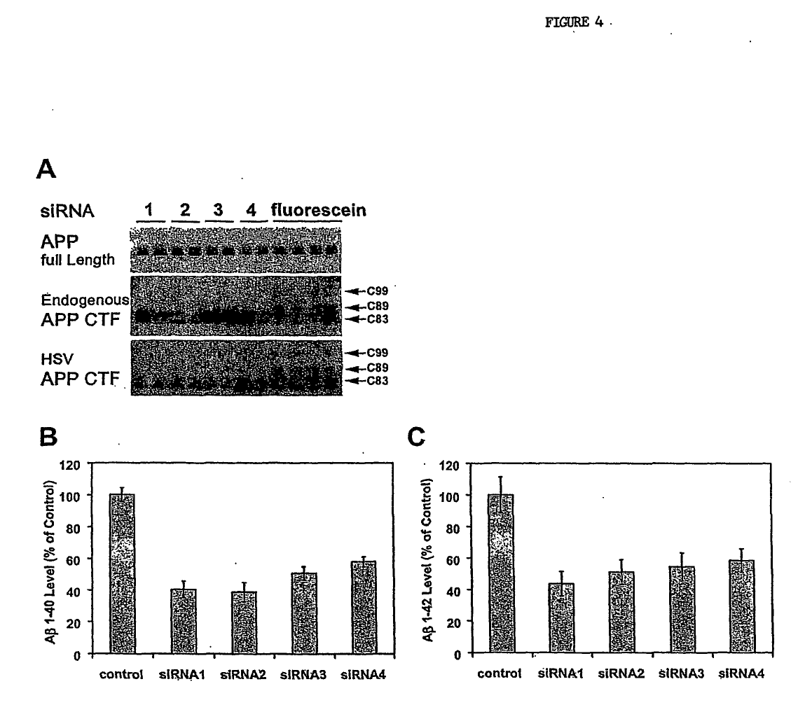

We have also tested the effects of these siRNAs in a hairpin structure. Sense and anti-sense sequences of BACE1 siRNA were cloned as a hairpin structure into a pSilencer vector using the protocol suggested by the manufacturer. The resulting pSilencer constructs were then introduced in conjugation with human BACE1 cDNA into CAD cells. Like siRNA duplexes, hairpin siRNAs can inhibit BACE1 protein expression (FIG. 3C). We also observed the highest efficacy of BACE1 protein knockdown with pSilencer-siRNA1 and pSilencer-siRNA3. In parallel experiments, primary cortical neurons were transfected with green fluorescence protein GFP) and pSilencer-siRNA constructs. We found that pSilencer-siRNA1 transfected neurons as indicated by GFP exhibited diminished BACE1 staining whereas neurons transfected with a non-specific siRNA sequence displayed robust BACE1 immunoreactivity (FIG. 4D, white arrows). Together, these results demonstrate that specific siRNAs can down regulate endogeneous BACE1 expression in primary cortical neurons.

### Example 5

### BACE1 siRNAs Effectively Reduced Aβ Secretion

As BACE1 cleavage of APP is the initial step for Aβ generation, we investigated the consequence of knocking-down endogeneous BACE1 expression on APP processing. Mouse primary cortical neurons were transfected with cognate BACE1 siRNA. In some experiments, neurons were infected with herpes simplex virus expressing human APP (HSV-APP) to facilitate detection of Aβ generation. 72 hours after siRNA treatment, we observed significant reduction in protein levels of BACE1 cleavage products of APP, C99/C89 in both non-infected and HSV-APP infected neurons (FIG. 4A). To determine if the reduced C99 levels impacted on secreted Aβ, we measured Aβ1-40 and 142 in cultured medium of neurons infected with HSV-APP. FIGS. 4B and 4C shows that BACE1 siRNAs effectively reduced Aβ secretion. There was a significant (40-60%) reduction of Aβ1-40 and Aβ1-42 secretion.

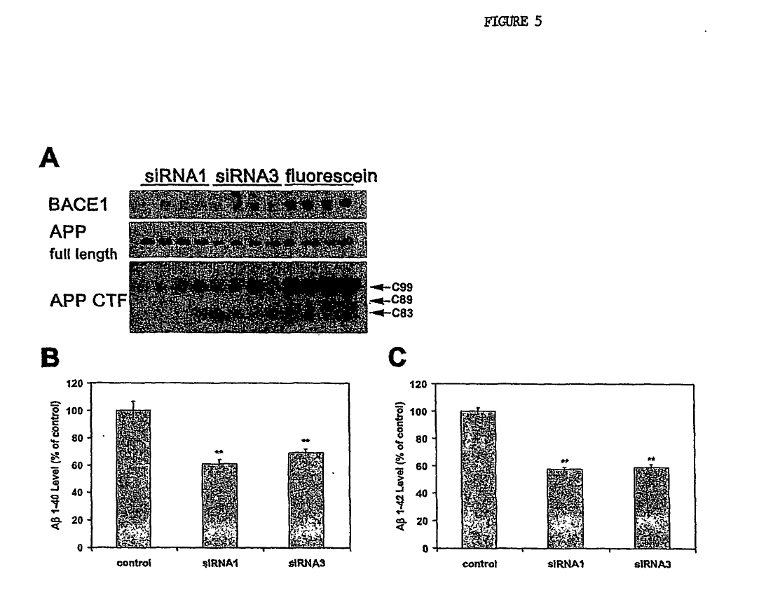

We have also tested the effects of siRNA on BACE1 expression and APP processing in primary cortical neurons from transgenic mice that express the Swedish double mutation (KM670/671NL) of the human APP (APPswe). Similarly, introducing BACE1 siRNA into APPswe cortical neurons lead to a decrease in BACE1 protein expression (FIG. 5A). Correspondingly, C99/89 generation (FIG. 5A) and Aβ secretion (FIG. 5B and 5C) was lowered.

Together, these results support the principle of reducing Aβ peptide generation using the BACE1 siRNA approach.

### Example 6

### Loss of BACE1 Using siRNA Elicits No Profound Cellular Defects

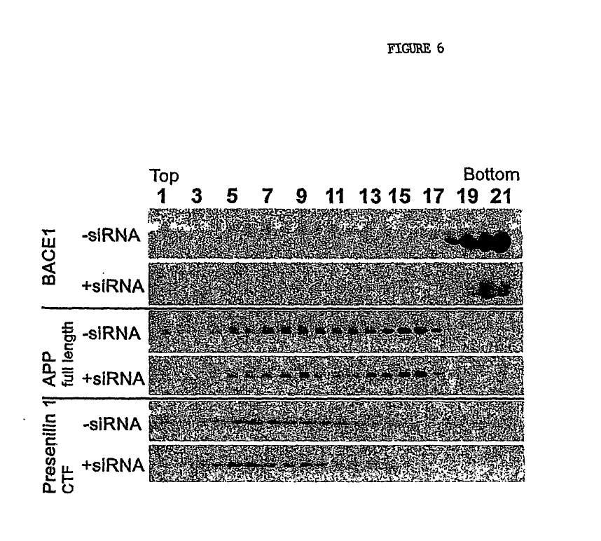

Although β-cleavage of APP is carried out by BACE1, little is known about the regulation of this cleavage event. It was suggested previously that APP, BACE1 and PS1 are assembled in the same transport vesicles [Kamal, 2001 #29], yet it is still not clear if BACE1 plays any role in trafficking and/or subcellular localization of APP and PS1. Here, we determined if suppression of BACE1 and subsequent APP CTF levels using siRNA changes APP and PS1 subcellular distribution. Control and BACE1 siRNA treated cortical neurons were fractionated using an iodixonal gradient (Optiprep), which separates organelle membranes according to vesicular density. As shown in FIG. 6, siRNA treatment efficiently lowered BACE protein levels. Importantly, there were no changes in APP and PS1 protein levels and subcellular distribution after siRNA treatment. These results further support the specificity of BACE1 siRNA. As loss of BACE1 using siRNA elicits no profound cellular defects, our data supports that BACE1 is an excellent therapeutic target for treatment of AD.

### Example 7

### BACE1 siRNAs Protect Against H₂O₂ Damage

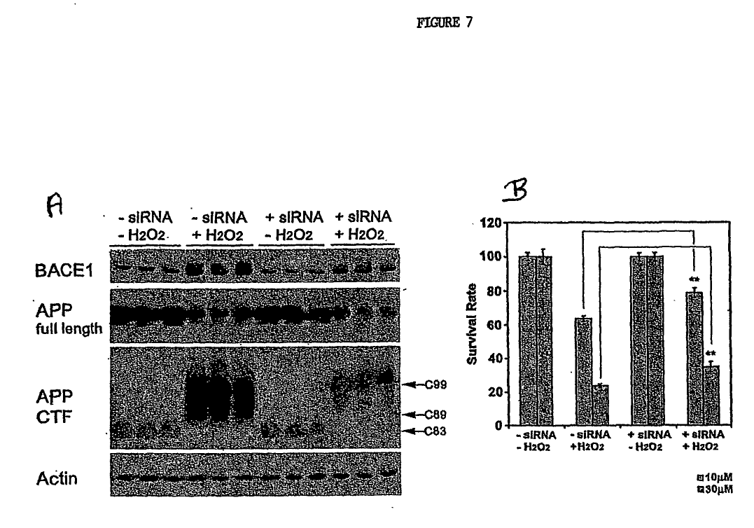

Oxidative stress has been implicated in pathogenesis and progression of AD. Many oxidation products including hydroxyl radicals (.OH) are found accumulating in AD brains. Thus, experiment system using hydrogen peroxide (H₂O₂) to induce oxidative stress has been widely applied for studying oxidative stress induced neurondegenerative responses. H₂O₂ been shown to induce intracellular accumulation of Aβ in neuroblastoma cells. It is currently unclear how H₂O₂ induces Aβ generation. Here, we treated primary cortical neurons with 10 μM H₂O₂ for 6 hours and found that both BACE1 protein and its products C99/89 were substantially increased accompanied with a decrease in full-length APP levels (FIG. 7A). Cell death was also induced after H₂O₂ treatment as examined by MIT assay (FIG. 7B). Interestingly, transfecting neurons with BACE1 siRNA lowered the BACE1 and C99/C89 induction levels following H₂O₂ treatment (FIG. 7A). Importantly, pretreatment with BACE1 siRNA prevented neurons from H₂O₂ induced cell death to a significant level (FIG. 7B). Together, these data shows that suppression of BACE1 expression protect neurons from oxidative stress induced cell death.

Thus, we demonstrated that oxidative stress induces BACE1 expression and subsequent C99/C89 generation. Interestingly, we found that pre-treating neurons with BACE1 siRNA lessens the neurotoxic effects of oxidative stress. This suggests that increased BACE1 expression by oxidative stress may triggers further neurodegeneration responses, which can be prevented by down-regulating BACE1, e.g., by using RNA interference.

### Example 8

### Materials and Methods for Examples 1-7

Antibodies and Reagents. Anti-APP was from Zymed Inc. Anti-BACE1 and anti-BACE2 were from Oncogene Science.

siRNA Preparation. siRNAs corresponding to BACE1 gene were designed as recommended (Elbashir et al. Nature, 411:494 and Elbashir et al. EMBO J. 20:6877), with 5′ phosphate, 3′ hydroxyl, and two base overhangs on each strand; they were chemically synthesized by Xeragon. The following sequences were used: siRNA1: 5′ gctttgtggagatggtgga 3′ (SEQ ID NO: 3); siRNA2: 5′ gacgctcaacatcctggtg 3′ (SEQ ID NO: 8); siRNA3 5′ tggactgcaaggagtacaa 3′ (SEQ ID NO: 13); siRNA4: 5′ ttggctttgctgtcagcgc 3′ (SEQ ID NO: 16). Annealing for duplex siRNA formation was performed as described (Elbashir et al. Nature, 411:494 and Elbashir et al. EMBO J. 20:6877).

Cell line and neuronal cell culture. Catecholaminergic cell lines (CAD cells) were cultured in Dulbecco's minimal essential medium (Invitrogen), supplemented with 10% fetal bovine serum, L-glutamine and 1% penicillin-streptomycin sulfate in a humidified 5% CO₂ incubator (Suri et al., 1993).

Primary cultures of embryonic mouse cortical neurons were prepared as described (Niethammer et al., 2000). In brief, dissociated embryonic neurons from E15 C57B1/6 or APPsw Tg pregnant mice were plated onto poly-D-lysine/laminin-coated 24-well plates or coverslips and maintained in neurobasal medium (Invitrogen) supplemented with B27 (Invitrogen), L-glutamine (Sigma-Aldrich) and 1% penicillin-streptomycin sulfate.

Generation of recombinant herpes simplex virus. Wildtype APP coding sequence were subcloned in to a replication-defective herpes simplex virus vector pHSVPrpUC. The resultant recombinant plasmid was packaged into virus particles in the packaging line 2-2 using the protocol previously described (Lim et al., 1996). The virus was then purified on a sucrose gradient, pelleted and resuspended in 10% sucrose and the titer of the recombinant virus was determined.

Immunocytochemistry. Primary cortical neurons from E15 mouse embryos were cultured at a density of 2×10⁵ cells/well in 24-well plates. Two day after plating, neurons were co-transfected with fluorescein RNA duplex and siRNA using Lipofectamine2000 (Invitrogen) for 1.5 hrs. In some experiment, one day in vitro culture were co-transfected with green fluorescence protein and pSilencer hairpin siRNA using calcium phosphate for 15 min. The medium was changed back to neurobasal after transfection. After 72 hr incubation, neurons were fixed in 4% paraformaldehyde for 30 min, blocked and permeabilized in 3% bovine serum albumin and 0.1% Triton in phosphate-buffered saline (PBS) for 20 min. Permeabilized neurons were incubated with primary antibodies for 1 hr at room temperature, and subsequently incubated with FITC or Alexa Fluor® 568 conjugated anti-mouse or anti-rabbit secondary antibodies (Molecular Probes). Images were captured using a Nikon inverted microscope linked to a DeltaVision deconvolution imaging system (Applied Precision).

C99/89 detection and Aβ measurement by ELISA assay. (1) Wildtype neurons expressing recombinant human APP. Primary cortical neurons from E15 mouse were cultured at a density of 4×10⁵ cells/well in 24-well plates. Two day in vitro neurons were transfected with siRNA using Lipofectamin 2000 for 1.5 hours. The medium was changed back to neurobasal after transfection. Neurons were incubated for another 48 hrs and then infected with herpes simplex virus expressing human APP for 24 hrs. Cell lysates were prepared for examining C99/C89 generation using Western blot analysis. Culture media was then collected and subjected to sandwich ELISA assay. Data were analyzed by t test using Prism (GraphPad). Differences were considered significant at p<0.05. (2) APPsw cortical Neurons. Primary cortical neurons from E15 APPsw Tg mice were cultured at a density of 4×10⁵ cells/well in 24-well plates. Two day in vitro neurons were transfected with siRNA using Lipofectamin 2000 for 1.5 hours. The medium was changed back to neurobasal after transfection. After 48 hr incubation, cultural medium were changed again and neurons were incubated for another 24 hrs. Subsequently, cell lysates and cultural medium were collected for examining C99/89 and Aβ generation.

H₂O₂ treatment of cell viability assay. Cell viability was examined using CellTiter 96® AQᵤₑₒᵤₛ One Solution Cell Proliferation Assay (Promega) according to the manufacturer's protocols.

Equivalents

It will be apparent to those skilled in the art that the examples and embodiments described herein are by way of illustration and not of limitation, and that other examples may be used without departing from the spirit and scope of the present invention, as set forth in the claims.

1. An siRNA molecule comprising a nucleotide sequence consisting essentially of a sequence set forth in SEQ ID NO: 1.

2. The siRNA of claim 1, wherein the nucleotide sequence consists of about 20 to 25 nucleotides.

3. The siRNA of claim 1, wherein the nucleotide sequence comprises SEQ ID NO: 3, 8, 13 or 18.

4. The siRNA of claim 1, wherein the nucleotide sequence consists essentially of SEQ ID NO: 3, 8, 13 or 18.

5. The siRNA of claim 4, wherein the nucleotide sequence consists of SEQ ID NO: 3, 8, 13 or 18.

6. An isolated nucleic acid encoding the sense strand, the antisense strand of both the sense and antisense strands of the siRNA molecule of claim 1.

7. An expression vector comprising the nucleic acid of claim 6.

8. A cell comprising the nucleic acid of claim 6.

9. A composition comprising at least two siRNAs of claim 1.

10. A composition comprising at least two nucleic acids of claim 6.

11. A method for reducing the level of BACE1 protein in a cell, comprising administering into the cell an siRNA molecule of claim 1.

12. A method for reducing the level of BACE1 protein in a cell, comprising administering into the cell a nucleic acid of claim 6.

13. The method of claim 11, comprising contacting the cell with the siRNA molecule.

14. The method of claim 11, wherein the cell comprises amyloid precursor protein (APP) and the method reduces the level of β amyloid (Aβ) peptide in the cell relative to a cell to which an siRNA or nucleic acid was not administered.

15. A method for preparing a pharmaceutical composition comprising combining an siRNA of claim 1 with a pharmaceutically acceptable carrier.

16. A method for treating or preventing Alzheimer's disease in a subject, comprising administering to the subject a therapeutically effective amount of an siRNA of claim 1, to thereby treat or prevent Alzheimer's disease.

17. The method of claim 16, wherein the administration of the siRNA reduces the level of Aβ peptides.

18. The method of claim 16, comprising administering the siRNA into senile plaques.

19. A method for protecting a cell against stress, comprising contacting the cell with or administering into the cell an siRNA molecule of claim 1, to thereby protect the cell from stress.

20. The method of claim 19, wherein stress is oxidative stress.


SEQUENCE LISTING (20 sequences)

SEQ ID NO: 1 - DNA, 1506 bp, Homo sapiens
  CDS (1)..(1506)
atggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtga

SEQ ID NO: 2 - protein, 501 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 3 - DNA, 19 bp, Homo sapiens
gctttgtggagatggtgga

SEQ ID NO: 4 - DNA, 19 bp, Mus musculus
gctttgtggagatggtgga

SEQ ID NO: 5 - DNA, 19 bp, Rattus sp.
gctttgtggagatggtgga

SEQ ID NO: 6 - DNA, 19 bp, Mus musculus
acttcttggctatggtgga

SEQ ID NO: 7 - DNA, 19 bp, Homo sapiens
acttcttggccatggtaga

SEQ ID NO: 8 - DNA, 19 bp, Homo sapiens
gacgctcaacatcctggtg

SEQ ID NO: 9 - DNA, 19 bp, Mus musculus
gacgctcaacatcctggtg

SEQ ID NO: 10 - DNA, 19 bp, Rattus sp.
gacgctcaacatcctggtg

SEQ ID NO: 11 - DNA, 19 bp, Mus musculus
gaaggtacagattcttgtg

SEQ ID NO: 12 - DNA, 19 bp, Homo sapiens
gaagctacagattctcgtt

SEQ ID NO: 13 - DNA, 19 bp, Homo sapiens
tggactgcaaggagtacaa

SEQ ID NO: 14 - DNA, 19 bp, Mus musculus
tggactgcaaggagtacaa

SEQ ID NO: 15 - DNA, 19 bp, Rattus sp.
tggactgcaaggagtacaa

SEQ ID NO: 16 - DNA, 19 bp, Homo sapiens
ttggctttgctgtcagcgc

SEQ ID NO: 17 - DNA, 19 bp, Mus musculus
ttggctttgctgtcagcgc

SEQ ID NO: 18 - DNA, 19 bp, Rattus sp.
ttggctttgctgtcagcgc

SEQ ID NO: 19 - DNA, 19 bp, Mus musculus
tgggctttgcagtgagtcc

SEQ ID NO: 20 - DNA, 19 bp, Homo sapiens
tgggcttcgcagcgagccc